(19) United States
(12) Patent Application Publication
(10) Pub. No.: US 2014/0276655 A1
(43) Pub. Date: Sep. 18, 2014

(54) DRAINAGE CATHETER WITH CUTTING TOOL

(71) Applicant: BOSTON SCIENTIFIC SCIMED, INC., MAPLE GROVE, MN (US)

(72) Inventors: JOHN F. MURRAY, MEDFIELD, CA (US); JEFFREY GRAY, SUDBURY, MA (US); NATHAN ZAMARRIPA, SUDBURY, MA (US)

(73) Assignee: BOSTON SCIENTIFIC SCIMED, INC., MAPLE GROVE, MN (US)

(21) Appl. No.: 14/204,786

(22) Filed: Mar. 11, 2014

Related U.S. Application Data
(60) Provisional application No. 61/780,839, filed on Mar. 13, 2013.

(51) Int. Cl. A61M 27/00

(52) U.S. Cl. CPC A61M 27/00
USPC 604/540

(57) ABSTRACT

Medical devices and methods for making and using the same are disclosed. An example medical device may include a drainage catheter. The drainage catheter may include an elongate catheter shaft having a lumen formed therein and a distal region proximal end. The distal region may have a plurality of openings formed therein. The openings may be in fluid communication with the lumen. The distal region may be configured to shift between a first configuration and a looped configuration. The catheter may also include an actuation member for shifting the distal region between the first configuration and the looped configuration. A cutting member may be disposed adjacent to the proximal end of the catheter shaft. The cutting member may be adapted to cut a portion of the actuation member.

### CROSS-REFERENCE TO RELATED APPLICATIONS

This application claims priority under 35 U.S.C. §119 to U.S. Provisional Application Ser. No. 61/780,839, filed Mar. 13, 2013, the entirety of which is incorporated herein by reference.

### TECHNICAL FIELD

The present disclosure pertains to medical devices, and methods for manufacturing medical devices. More particularly, the present disclosure pertains to drainage catheter.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a drainage catheter. The drainage catheter may include an elongate catheter shaft having a lumen formed therein, a distal region, and a proximal end. The distal region may have a plurality of openings formed therein. The openings may be in fluid communication with the lumen. The distal region may be configured to shift between a first configuration and a looped configuration. The catheter may also include an actuation member for shifting the distal region between the first configuration and the looped configuration. A cutting member may be disposed adjacent to the proximal end of the catheter shaft. The cutting member may be adapted to cut a portion of the actuation member.

Another example medical device may include an elongate catheter shaft having a lumen formed therein, a distal region, and a proximal end. The distal region may have one or more openings formed therein. The openings may be in fluid communication with the lumen. The distal region may be configured to shift between a first configuration and a looped configuration. A hub may be secured to the proximal end of the catheter shaft. The medical device may also include a suture for shifting the distal region between the first configuration and the looped configuration. The suture may be coupled to the distal region of the catheter shaft and may extend to a position adjacent to the hub. A cutting member may be coupled to the hub. The cutting member may be adapted to cut a portion of the suture.

An example method for using a drainage catheter may include providing a drainage catheter. The drainage catheter may include an elongate catheter shaft having a lumen formed therein, a distal region, and a proximal end. The distal region may have a plurality of openings formed therein. The openings may be in fluid communication with the lumen. The distal region may be configured to shift between a first configuration and a looped configuration. The catheter may also include an actuation member for shifting the distal region between the first configuration and the looped configuration. A cutting member may be disposed adjacent to the proximal end of the catheter shaft. The cutting member may be adapted to cut a portion of the actuation member. The method may also include disposing the distal region of the catheter shaft within a body lumen, actuating the actuation member to shift the distal region to the looped configuration, and cutting a portion of the actuation member with the cutting member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is an illustrative diagram of a drainage catheter according to one embodiment of the present disclosure;

FIG. 2 is a cross-sectional view of an exemplary collar according to one embodiment of the present disclosure;

FIG. 3 is an illustrative diagram of the drainage catheter disposed within a patient in an unlocked position according to one embodiment of the present disclosure;

FIG. 4 is an illustrative diagram of the drainage catheter disposed within a patient in a locked position according to one embodiment of the present disclosure;

FIG. 5 illustrates components of a drainage catheter hub in a locked position;

FIG. 6 is an exploded view of drainage catheter hub components;

FIG. 7 is an illustrative diagram of a hub assembly of the drainage catheter according to one embodiment of the present disclosure;

FIG. 8 is an illustrative diagram of the hub assembly of FIG. 4 with a jacket according to one embodiment of the present disclosure;

FIG. 9 is a side view of a portion of an example drainage catheter;

FIG. 10 illustrates an example key member;

FIG. 11 illustrates another example key member;

FIG. 12 illustrates another example key member;

FIG. 13 illustrates an example collar member;

FIG. 14 is a cross-sectional view of a portion of an example hub;

FIG. 15 is a partial cross-sectional view of a portion of another example hub in a first configuration; and

FIG. 16 is a partial cross-sectional view of a portion of the example hub shown in FIG. 15 in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Typically, drainage catheters are tubular, flexible conduits percutaneously inserted into a fluid collection within the viscera. Common applications of drainage catheters include abscess, biliary, and nephrostomy drainage resulting from the body's temporary inability to naturally drain these fluid collections. A drainage catheter may be introduced over a stiffening cannula using either a direct trocar stick or a Seldinger technique, over a guidewire.

To inhibit catheter movement, a pigtail loop or other retention structure is often formed at the catheter's distal end. The loop, once formed, engages surrounding tissue, such as the inner walls of a lumen or organ, preventing the catheter from displacing due to accidental tugging or pulling.

Some catheters include a pre-formed pigtail loop at their distal end. Before placing this catheter in the body, a rigid wire is inserted to straighten out the loop. Once placed, the rigid wire is removed. Alternatively, the pigtail loop may be formed after the instrument is inserted at the desired location. In such catheters, a suture, fixed to the catheter's distal end, extends along the catheter's length, and exits from its proximal end. By drawing the suture proximally, the distal tip of the catheter is forced to curl into a pigtail formation. A proximal suture portion is then secured to hold it in place and retain the loop shape at the distal end of the catheter.

Many such lockable pigtail loop catheters are available today. Some lockable catheters include a locking mechanism that requires a separate unlocking tool. If the unlocking tool is misplaced, it may be difficult to release such sutures and disengage the catheters. Other lockable catheters do not require external unlocking tools, rendering them susceptible to being inadvertently unlocked.

Therefore, there remains room for improvement and/or alternatives in providing tamper-resistant structures for drainage catheters.

Embodiments of the present disclosure introduce drainage catheters used to drain viscera such as the kidneys, bladder, abdominal cavity, stomach, and biliary system. These catheters may include a retention element such as a pigtail loop with a novel suture-locking hub. The locking hub minimizes tampering risks by providing a retention system, which is difficult to inadvertently open, yet does not require any additional unlocking tools. The locking hub may include a base attached to the proximal end of the catheter. A flexible member such as a suture may be connected to the distal end of the elongate member and may extend through the elongate member towards the hub. In the hub, the suture may exit from a small opening in the base. Proximally pulling the suture thread may actuate the retention element forming the pigtail loop. A collar may cover the base, retaining the suture between the collar's inner surface and the base's outer surface. A screw-fit lock including a screw threading on the outer surface of the base may engage a corresponding screw thread on the inner surface of the collar to lock the base and the collar. The base's outer surface and the collar's inner surface may also positively engage when the screw threads are engaged to provide further retention. The base may taper distally at a particular angle, while the collar may taper at a different angle. Moreover, the collar may taper at a sharper angle than the base making the tapered collar diameter slightly lesser than the corresponding diameter of the base.

The following exemplary embodiments illustrate the retention element as a pigtail loop. It will be understood, however, that this depiction is merely exemplary and not meant to limit the scope of the present disclosure. For example, the retention element may be a malecot element, an element having struts or any other element that can be expanded, contracted or otherwise actuated to change configuration to secure the catheter within a patient.

FIG. 1 illustrates an exemplary drainage catheter including an elongated body member 102, an elongate flexible member 104 (or flexible member 104), and a hub 106.

The elongated body member 102 includes a central lumen 108 extending from its distal end 109 to its proximal end 110. The member 102 may have any cross-sectional shape, such as circular, rhombic, rectangular, oval, semicircular, or any other suitable shape. Moreover, the elongated member diameter may vary depending on the quantity of fluid to be extracted, fluid density, the size of the body cavity or cannula used to guide the elongated member 102 towards the desired location. Further, the elongated body member 102 may have a uniform cross-section or diameter from its distal 109 to proximal end 110. Alternatively, the cross-section and diameter may vary through its length.

The elongated body member's distal portion includes one or more apertures 112 to facilitate fluid flow from the patient's body to the central lumen 108. These apertures can be adapted to fit the needs of a given application. For example, the number of apertures 112 and their shape may vary based on the nature of the fluid. For example, if the fluid is viscous, the apertures 112 may be larger or more numerous. Alternatively, if the fluid has lower density, the apertures 112 may be smaller or lesser in number. It will be understood that the size and number of apertures are not restrictive, and elongated members with any size and number of apertures are well within the scope of the present disclosure.

Because the elongated body member 102 is within a patient's body for short or extended periods, the device is made of non-allergic or biocompatible material. Such materials include, for example, silicones and polyurethanes. It will be understood that any other suitable material may just as easily be used. In one embodiment, the elongated member 102 may be coated with an anti-bacterial coating to inhibit bacterial growth on its surface. The anti-biotic coating may contain an inorganic anti-biotic agent, disposed in a polymeric matrix that adheres the anti-biotic agent to the elongate member's surface. Further, a drug releasing coating may also be applied to the outer surface of the elongated member 102, assisting in healing. In another embodiment, the elongated member 102 includes a lubricious coating to facilitate convenient insertion.

The flexible member 104 may be coupled to a distal portion of the elongated body member 102, and it may exit the elongated body member 102 through a distal opening 114. From here, the elongated flexible 104 member may reenter the central lumen 108 through a second opening 116 disposed at a little distance from the distal end 109. Within the central lumen 108, the flexible member 104 travels from the distal end to the proximal end, and then extends out of the elongated member 102. This member may be a suture thread made of nylon or other similar material of comparable strength. Alternatively, the member may be a thread or a flexible metal wire.

In another embodiment, instead of attaching to the distal end 109 of the elongated member 102, the flexible member 104 may be coupled to the elongate member-hub junction. From this junction, this member extends distally through the central lumen 108 towards the distal end 109. From there, it exits the lumen 108 through the distal opening 114 and reenters the lumen from the second opening 116. It then returns proximally through the central lumen 108 towards the hub 106. To attach the flexible member 104 to the elongate member's distal end 109 or the elongate member-hub junction, suitable coupling techniques, such as tying, gluing, or piercing, may be used.

The hub 106 includes a base 118 and a collar 120. The base 118 is a cylindrical tube including a proximal end 119, a distal end 121, an internal passageway 122, a side lumen 124, and an external surface 126. Its distal end 121 is coupled to the elongated body member's proximal end 110, and its internal passageway 122 (extending along its length) is coaxial with the central lumen 108. The external surface 126 of the base 118 tapers from its proximal end 119 to its distal end 121, and it includes an engagement element 128. In one embodiment, only a portion of the base 118 tapers. In this case, the tapering may begin distal of the engagement element 128 and extend to the distal end 121. The side lumen 124 extends from the internal passageway 122 to the external surface 126. This lumen provides a path for the flexible member 104 to exit the base 118.

A mating surface 129 is present on the external surface 126. This portion may begin distal of the engagement element 128 and extend up to the base's distal end 121. Alternatively, the mating surface 129 may extend for a portion of the external surface 126 distal of the engagement element 128. In one embodiment, the mating surface 129 tapers proximally to distally.

In addition to the elements described in the immediately preceding section, the base 118 may further include screw threads 130 at its proximal end 119. These screw threads 130 attach peripheral instruments to the drainage catheter 100 such as collection bags, drainage tubes, syringes, and so on. It will be understood that instead of the threaded arrangement, other means may also be employed to attach peripheral equipment to the hub 106. For example, snap fit arrangement may be employed.

Base 118 may be composed of a semi-rigid material such as molded biocompatible plastic, nylon, polyethylene, ethylene-vinyl acetate co-polymer, or a shape memory metal, such as nitinol. Alternately, a semi-rigid compliant member, such as rubber may form the base 118.

Collar 120 is a hollow cylindrical structure that snuggly fits around the base 118 when the distal end 109 is placed at the desired location within a patient's body. FIG. 2 is a cross-sectional view of the collar 120. The collar may include a tapered inner surface 132 and an outer surface 134. In at least some embodiments, the inner surface 132 and/or a portion thereof may take the form of a mating surface that is configured to mate with the mating surface 129 of the base 118. Moreover, like the external surface 126 of the base 118, the collar's inner surface 132 may also taper from its proximal end to its distal end, as illustrated, or vice versa. The tapering angle, however, differs from the base's tapering angle. In one embodiment, the collar's tapering angle is greater than that of the base 118. Further, the collar's inner surface 132 includes a counter element 136 that may mate with the engagement element 128 when the collar 120 is placed around the base 118. The collar 120 also includes a side lumen 138 that extends from the inner surface 132 to the outer surface 134. The flexible member 104 may pass through this side lumen 138. The mating surface of the inner surface 132 may extend from the counter element 136 to the collar's distal end. Alternatively, the mating surface may begin distal of the counter elements 136 and its length may be equal to the length of the mating surface 129 of the base 118.

Engagement element 128 and counter element 136 are shown as a helical male thread and a corresponding female thread. These threads may be mating threads or they may have slightly different pitches so that the force between the threads may add to the force between the collar 120 and the base 118 when locked together. The structure of the engagement element 128 and the counter element 136 may vary in different embodiments of the drainage catheter 100. For example, these elements may be helical male and female threads, members of a snap-fit assembly, male and female luer-lock elements, protrusions and notches, ratcheting members, etc. It will be understood that any suitable engagement element 128 and counter element 136 may be utilized without departing from the scope of the present disclosure.

Similar to the base, the collar 120 may also be formed of a semi-rigid or rigid material, such as nylon, rubber, biocompatible plastic and so on.

The structural features of the drainage catheter 100 are described with reference to FIGS. 1-2. The following figures (FIGS. 3-4) describe the catheter's functional features, including a method of locking the flexible member 104 in the hub 106. FIG. 3 illustrates the drainage catheter 100 in a first position, with hub 106 in an unlocked state; and FIG. 4 illustrates the drainage catheter 100 in a second position, with hub 106 in a locked state. The figures, in succession, illustrate a process of inserting the drainage catheter 100 in a patient's body, forming a pigtail loop, and securing the flexible member 104 to maintain the pigtail loop.

Initially, to insert the distal end 109 within a cavity 201 in the patient's body 203, the elongated member 102 is straightened with a stiffening stylus 202 disposed within its central lumen 108. A guide wire may also be inserted in the central lumen 108 to guide the catheter to the desired location. The straightened elongated member 102 is then advanced to the cavity 201 using any suitable procedure such as percutaneous insertion, or insertion through a body cavity. Once in place, the guide wire and stiffening stylus 202 are withdrawn by pulling them back out of the catheter's proximal end 204.

During this insertion procedure, the collar 120 is not secured over the base 118. It may instead rest around the proximal portion of the elongated member 102. Moreover, the flexible member 104 extends from the elongated member 102 to the base 118. From the base 118, the flexible member 104 exits via the side lumen 124, enters the collar 120, and exits the collar 120 through the collar's side lumen 138. A portion of the flexible member extends out from the collar's side lumen 138.

Once the catheter is in place, a pigtail loop is formed and secured. This operation is illustrated in FIG. 4. An operator may pull the flexible member 104 extending from the side lumen 138 to draw the distal end 109 of the elongated member 102 proximally, forcing it to curl up and form the pigtail loop 206. To lock the pigtail loop 206, the operator, while pulling the flexible member 104 taut with one hand, may move the collar 120 towards the base 118 with the other hand. The collar 120 is pushed until the collar's counter element 136 contacts the engagement element 128.

In one embodiment, the engagement element 128 and the counter element 136 are helical external and internal screw threads, respectively. Once contacted, the collar 120 may be twisted, to engage the external screw thread in the internal thread, locking the base 118 and the collar 120. Further, the helical external and internal screw threads may extend less than 360° around the base's external surface, and their major diameter may be lesser than their lead.

The collar length and its taper angle may be selected such that when the screw thread of the collar 120 reaches the helical screw thread on the base 118, the inner diameter of the distal end of the collar 120 is equivalent to the outer diameter of the base portion that is in contact with the collar's distal end. So, when the collar 120 is screwed on the base 118, the collar 120 twists proximally, forcing the inner surface 132 of the collar 120 into a tight fit over the mating surface 129 of the base 118 and sealing the space between collar 120 and base 118. This mechanism introduces a double lock. Because of the force exerted by the tight fit between the mating surfaces of the base 118 and collar 120, it is difficult to disengage the hub 106. Moreover, even if the engagement element 128 wears off over time, or is inadvertently disengaged, considerable force may be needed to separate the collar 120 and the base 118 because of the mating force between their mating surfaces. Therefore, the pigtail loop 206 stays intact even if the device is tampered with.

FIG. 5 is a detail view of hub 106 in the locked position. Here, a portion of the flexible member 104 (between the side lumen 124 and the side lumen 138) is trapped between the collar 120 and base 118, preventing any movement. It will be understood that once locked, the side lumens should not overlap one another. The separation between the two lumens 124, 138 determines the length of flexible member 104 trapped between the base 118 and the collar 120. To reduce the strain on the flexible member 104, it may be desirable to have a relatively lengthy portion between the lumens. Therefore, the lumens may be placed as far away from each other as possible.

In the locking position, side lumens 124 and 138 may be in a variety of relative positions. For example, the lumens may lie in the same longitudinal plane, but be circumferentially separated from one another. A second example position occurs with the lumens lying in the same circumferential plane, but in different longitudinal planes. In another exemplary embodiment, the lumens could lie in different circumferential and longitudinal planes along the surface of the base 118 and the collar 120. The side lumens 124, 138 may be positioned anywhere along the external surface of the base 118 and collar 120.

It will be understood that the helical screw thread assembly illustrated in the figures is merely exemplary. The engagement element 128 and counter element may be any suitable engaging arrangement without departing from the scope of the present disclosure. For example, the engagement and counter elements may be elements of a snap fit assembly or a luer-lok assembly. Alternatively, the engagement element 128 may be multiple spring-loaded angular projecting flanges while the counter element may carry similarly shaped grooves. When the collar 120 travels proximally over the base 118 the projections may engage the grooves locking the collar 120 to the base 118. To unlock the hub 106, the base 118 may include a spring-release button. Pressing this button contracts the spring in the flanges, pulling the flanges towards the base's surface, thereby releasing them from the groove, and unlocking the hub 106. To ensure that the release button is not accidently pressed, it may be embedded in the base's body between the inner and out surface, similar to reset buttons present on most electronic devices. Any other suitable engagement mechanism may be contemplated and is within the scope of the present disclosure.

To complete placement, the excess flexible member 104 extending from the collar's side lumen 138 may be trimmed. With the flexible member 104 trimmed, if the catheter 100 is accidentally unlocked, the pigtail loop 206 at the distal end may straighten out, and the proximal end of the flexible member 104 may be lost within the central lumen 108. It can be difficult to extract the flexible member 104 in this situation. To overcome this difficulty, the proximal portion of the flexible member 104 may be coiled around the base 118 before the collar 120 is locked in place and the flexible member 104 is trimmed. This excess flexible member 104 between the base 118 and the collar 120 allows operators to unlock the hub 106, straighten the pigtail loop 206, and manipulate the placement of the elongated member 102 without losing the flexible member 104.

It will be understood that various alternatives may be contemplated for the hub 106. FIG. 6 illustrates one such alternative. Here, the hub 106 includes a jacket 402 that covers both the base 118 and the collar 120, in an assembled condition. In one embodiment, the jacket 402 may be made of a pliable material that deforms under pressure or that may change its shape according to underlying instruments. Such material may include rubber, polymer, nylon, etc. A flexible jacket 402 may be used because the external surface of the hub 106 is uneven, and fitting a rigid jacket over this surface may pose difficulties.

Further, the jacket 402 includes a cavity 404 coaxial with the base's internal passageway 122. The jacket 402 is pulled proximally over the collar 120 and base 118, to prevent accidental tampering. In the illustrated embodiment, the jacket 402 tapers distally and extends beyond the distal ends of the base 118 and the collar 120. Alternatively, a distal portion of the jacket 402 could taper, while its proximal portion maintains a uniform cross-section.

In another embodiment, the jacket 402 may be made of two equal and identical shaped portions, obtained by, for example, slicing the jacket longitudinally. These longitudinal portions align to form the jacket. In an embodiment, the two longitudinal portions may cover the collar and base such that the two ends of the longitudinal portion touch each other. Subsequently, the portions may be secured using known securing mechanisms such as snap-fitting, locking, glue, etc. Alternatively, the two portions may be hingedly attached from one end, while the other end may swing open. In this implementation, when the two free ends contact each other, they form a hollow space within to cover the hub. The jacket may open around the hinge to place over the collar and base. Once placed, the jacket 402 may be closed, and suitable mechanisms may be included to lock the open edges of the jacket 402. In these embodiments, the internal surface of the jacket 402 may be shaped like the external hub surface. As the jacket 402 opens laterally, it can easily fit over the hub 106, and, therefore, it may not be made of a flexible or elastic material. Instead, the jacket 402 may be formed of any suitable rigid or semi-rigid material, such as plastic, metal, polymer, and so on.

As illustrated in FIG. 6, the base's side lumen 124 is distal of the engagement element 128. This placement minimizes potential interference between the flexible member 104 and any peripheral instruments attached to the proximal end of the hub 106. Placing the side lumen 124 distal of the engagement element 128, however, may result in fluid leakage through that lumen. To prevent such leakage, the side lumen 124 may include a sealing member 406. Any sealing member, such as a pliant plug having a threadable hole may be used. Likewise, insoluble materials with self-sealing properties may seal the lumen. Such materials include silicone, wax, rubber, or latex. In some embodiments, the collar's side lumen 138 may also be sealed in a similar manner. Alternatively, a deformable sealing material may be disposed between the base 118 and the collar 120, providing a similar sealing function when locked together.

FIG. 7 is a detail view of the hub 106 of FIG. 1 with the jacket 402. Here, the jacket 402 lies on the proximal portion of the elongated member 102 distal of the collar 120 and base 118. When the hub 106 is assembled for locking, the collar 120 engages with the base 118 and locks the flexible member 104 in position. The jacket 402 then slips over the locked base and collar assembly, isolating it from external conditions.

FIG. 8 is a sectional view of the hub 106 and the jacket 402 in the locked state. The internal lumens, passageways, and surfaces of the elongated member 102, base 118, collar 120, and jacket 402 are visible in this view. The proximal diameter of the elongated member 102 is sized to match the distal diameter of the base 118, providing a smooth transition from one lumen to the other. For instance, the elongated member 102 and the base 118 may be fused or bonded to one another. The internal passageway 122 may gradually increase in diameter from the distal end 121 to the proximal end 119. Alternatively, the passageway 122 may remain uniform throughout the length of the base 118, or it may taper towards the proximal end, neither case departing from the scope of the present disclosure.

FIG. 8 illustrates the inner surface 132 of the collar 120 along with the mating surface 133, the counter element 136, and the side lumen 138. The inner surface 132 of the collar 120 tapers from the proximal end to the distal end or vice versa. As described previously, the collar's taper angle may differ from that of the base 118. For example, the tapering angle of the collar's inner surface 132 may be greater than the tapering angle of the base's outer surface 126. Because the tapering angle is greater, the collar's mating surface 133 tightly fits around the base's mating surface 129 providing an airtight surface lock between the collar 120 and base 118.

The internal surface of the jacket 402, as depicted, follows the contour of the collar 120 up to the distal end of the collar 120. From there on, the jacket 402 tapers at a suitable angle towards the elongated member 102. The tapering angle determines the length of the jacket 402 distal of the collar 120; the sharper the angle, the shorter the jacket 402.

FIG. 8 also illustrates the embodiment where the flexible member 104 is attached to the junction between the elongated member 102 and the base 118. Here, the flexible member 104 travels distally through the central lumen 108 towards the distal end 109, threads between the distal opening 114 and second opening 116, and returns through the central lumen 108 toward the hub 106. In the hub 106, the flexible member 104 exits from the side lumens of both the base 118 and the collar 120.

As indicated above, when the flexible member 104 is secured with the collar 120, it may be desirable to trim off an excess portion of the flexible member. In order to do so, a clinician may use a scalpel, scissors, or the like. Because these cutting tools are typically not part of the catheter or catheter system, the clinician will need to provide these cutting tools separately. The present disclosure pertains to medical devices and/or systems that include structural features that may be used to cut or sever a portion of the flexible member 104 (e.g., any excess portion of the flexible member 104). These cutting structures may be a portion of medical device that is adapted to cut the flexible member 104, an accessory that is incorporated into the medical device or otherwise associated therewith and can be used to cut the flexible member 104, or the like.

FIG. 9 illustrates a portion of example medical device 500 similar to other devices disclosed herein. In at least some embodiments, the medical device 500 may take the form of a drainage catheter including any of the features disclosed herein. For example, the medical device 500 may include a catheter shaft 502. The catheter shaft 502 may be similar to the catheter shaft or elongate body 102 as described above. For example, the catheter shaft 502 may include a plurality of apertures formed therein (e.g., similar to the apertures 112) that may be similarly used for drainage. Furthermore, the catheter shaft 502 may be configured to shift between a first generally straightened configuration and a second generally curved or looped configuration. A suture or flexible member (e.g., similar to the flexible member 104) may be utilized to shift the catheter shaft 502 between configurations.

A hub 506 may be coupled to the catheter shaft 502. In general, the hub 506 may be used in a manner similar to other hubs disclosed herein. For example, a clinician may pull on the flexible member (e.g., the flexible member 104) to shift the catheter shaft 502 into the looped configuration and then utilize the hub 506 to secure the flexible member relative to the hub 506 and, thus, hold the catheter shaft 502 in the looped configuration.

In at least some embodiments, the hub 506 may include a collar 534, similar to other collars disclosed herein, that may be slidable along the hub 506. In at least some embodiments, the collar 534 may have an opening 538 formed therein. The opening 538 may be a suture opening or otherwise an opening for the flexible member (e.g., the flexible member 104). In use, the collar 534 may be slid along the hub 506 in a manner such that the flexible member (e.g., the flexible member 104) is secured and “locked” therebetween. The loose end or excess portion of the flexible member may extend through the opening 538. However, this is not required.

A key member 540 may also be coupled to the hub 506. In general, the key member 540 may be disposed along a portion of the hub 506 where the collar 534 may slide. Thus, the key member 540 may form a physical barrier that prevents undesired movement of the collar 534 relative to the hub 506. When movement of the collar 534 is desired, the key member 540 may be removed from the hub 506, thereby opening space for the collar 534 to move.

In at least some embodiments, the key member 540 may include a structural feature that is configured to allow a portion of the flexible member to be trimmed or cut. For example, FIG. 10 illustrates that the key member 540 may include a cutting member 546. In some embodiments, the cutting member 546 may take the form of a narrowed notch or opening. In use, the key member 540 may be removed from the hub 506 and the flexible member may be pulled into the notch/cutting member 546 and trimmed. The key member 540 may also include a tab portion 542 and one or more legs such as legs 544a/544b. The legs 544a/544b may be utilized to help releasable secure the key member 540 to the hub 506.

FIG. 11 illustrates another example key member 640 that may be similar in form and function to other key members disclosed herein. The key member 640 may include a body portion 642 having a cutting member 646 formed therein. Rather than having legs 544a/554b like key member 540, key member 640 simply has a base that is configured to fit into a socket or opening in a suitable hub.

FIG. 12 schematically illustrates another example key member 740 that may be similar in form and function to other key members disclosed herein. The key member 740 may include a body portion 742 having a cutting member 746 formed therein. According to this embodiment, the cutting member 746 may have a flap-like structure. In at least some embodiments, the flap-like structure may resemble the cutting edge on a dental floss dispenser.

FIG. 13 schematically illustrates a portion of another example collar 834 that may be utilized with any of the devices disclosed herein. The collar 834 may include an opening 838 formed therein. A cutting member or cutting region 846 may be defined in the opening 838. Thus, the opening 838, itself, may be utilized to cut any excess portions of the flexible member.

FIG. 14 illustrates a portion of another example medical device 900 that may be similar to other medical devices disclosed herein. In at least some embodiments, the medical device 900 may take the form of a drainage catheter including any of the features disclosed herein. The medical device 900 may include a catheter shaft 902. A hub 906 may be coupled to the catheter shaft 902. A key member 940 may be coupled to the hub 906. However, this is not required. In at least some embodiments, the hub 906 may include a collar 934. In at least some embodiments, the collar 934 may have a projection or thread 950 that may be configured to engage an opening or thread 952 of a base member 948 of the hub 906. The collar 934 may include a cutting member 946. In at least some embodiments, the cutting member 946 may take the form of a cutting edge or blade disposed along an inner surface of the collar 934. The cutting member 946 may be used to trim off a portion of the flexible member. This may include bringing the thread 950 of the collar 934 into engagement with the thread 952 of the base member 948. In doing so, the collar 934 may be rotated. Rotation may result in the cutting member 946 coming into contact with the flexible member and, thus, the trimming off of any excess portions of the flexible member.

FIGS. 15-16 illustrate a portion of another example medical device 1000 that may be similar to other medical devices disclosed herein. In at least some embodiments, the medical device 1000 may take the form of a drainage catheter including any of the features disclosed herein. The medical device 1000 may include a catheter shaft 1002. A hub 1006 may be coupled to the catheter shaft 902. The hub 1006 may include a lever or handle 1040. The lever 1040 may include a securing member 1054 and a cutting member 1052 that are configured to secure and trim, respectively, a flexible member 1004. For example, actuating the lever 1040 may cause the cutting member 1052 to come into contact with and trim away an excess portion 1004′ of the flexible member 1004 as shown in FIG. 16. In doing so, the flexible member 1004 may be secured within the hub 1006 between the securing member 1054 and a securing surface 1056 of the hub 1006.

The materials that can be used for the various components of devices disclosed herein may include those commonly associated with medical devices. For example, the devices disclosed herein and/or the components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A drainage catheter, comprising:
an elongate catheter shaft having a lumen formed therein, a distal region, and a proximal end;
wherein the distal region has a plurality of openings formed therein, the openings being in fluid communication with the lumen;
wherein the distal region is configured to shift between a first configuration and a looped configuration;
an actuation member for shifting the distal region between the first configuration and the looped configuration; and
a cutting member disposed adjacent to the proximal end of the catheter shaft, the cutting member being adapted to cut a portion of the actuation member.

2. The drainage catheter of claim 1, wherein a hub is attached to the proximal end of the catheter shaft.

3. The drainage catheter of claim 2, wherein the cutting member is coupled to the hub.

4. The drainage catheter of claim 2, wherein the hub includes a key member and wherein the cutting member is coupled to the key member.

5. The drainage catheter of claim 4, wherein the cutting member includes a cutting notch disposed along the key member.

6. The drainage catheter of claim 4, wherein the cutting member includes a cutting flap disposed along the key member.

7. The drainage catheter of claim 2, wherein the hub includes a slidable collar, and wherein the cutting member is disposed along the slidable collar.

8. The drainage catheter of claim 7, wherein the collar includes an aperture and wherein the cutting member includes a narrowed region of the aperture.

9. The drainage catheter of claim 7, wherein the cutting member is disposed along an inner surface of the collar.

10. The drainage catheter of claim 2, wherein the hub includes a rotatable lever and wherein the cutting member is disposed along the rotatable lever.

11. The drainage catheter of claim 1, wherein the actuation member includes a suture.

12. A medical device, comprising:
an elongate catheter shaft having a lumen formed therein, a distal region, and a proximal end;
wherein the distal region has one or more openings formed therein, the openings being in fluid communication with the lumen;
wherein the distal region is configured to shift between a first configuration and a looped configuration;
a hub secured to the proximal end of the catheter shaft;
a suture for shifting the distal region between the first configuration and the looped configuration;
wherein the suture is coupled to the distal region of the catheter shaft and extends to a position adjacent to the hub; and
a cutting member coupled to the hub, the cutting member being adapted to cut a portion of the suture.

13. The medical device of claim 12, wherein the hub includes a key member and wherein the cutting member is coupled to the key member.

14. The medical device of claim 13, wherein the cutting member includes a cutting notch disposed along the key member.

15. The medical device of claim 13, wherein the cutting member includes a cutting flap disposed along the key member.

16. The medical device of claim 12, wherein the hub includes a slidable collar, and wherein the cutting member is disposed along the slidable collar.

17. The medical device of claim 16, wherein the collar includes an aperture and wherein the cutting member includes a narrowed region of the aperture.

18. The medical device of claim 16, wherein the cutting member is disposed along an inner surface of the collar.

19. The medical device of claim 12, wherein the hub includes a rotatable lever and wherein the cutting member is disposed along the rotatable lever.

20. A method for using a drainage catheter, the method comprising:
providing a drainage catheter, the drainage catheter comprising:
an elongate catheter shaft having a lumen formed therein, a distal region, and proximal end,
wherein the distal region has a plurality of openings formed therein, the openings being in fluid communication with the lumen,
wherein the distal region is configured to shift between a first configuration and a looped configuration,
an actuation member for shifting the distal region between the first configuration and the looped configuration, and
a cutting member disposed adjacent to the proximal end of the catheter shaft, the cutting member being adapted to cut a portion of the actuation member;
disposing the distal region of the catheter shaft within a body lumen;
actuating the actuation member to shift the distal region to the looped configuration; and
cutting the portion of the actuation member with the cutting member.
